(19) United States
(12) Patent Application Publication
(10) Pub. No.: US 2013/0109049 A1
(43) Pub. Date: May 2, 2013

(54) Method of Establishing a Fungal Nail Infection

(71) Applicants: Deborah O'Neil, Craibstone (GB); Derry Mercer, Craibstone (GB); Colin Stewart, Craibstone (GB)

(72) Inventors: Deborah O'Neil, Craibstone (GB); Derry Mercer, Craibstone (GB); Colin Stewart, Craibstone (GB)

(73) Assignee: NOVABIOTICS LIMITED, Craibstone, Aberdeen (GB)

(21) Appl. No.: 13/517,230

(22) PCT Filed: Dec. 20, 2010
(86) PCT No.: PCT/GB10/02313

§ 371 Date: Aug. 22, 2012

(30) Foreign Application Priority Data
Dec. 22, 2009 (GB) 0922403.1

(51) Int. Cl. C12Q 1/18; C12N 1/16; C12N 1/14

(52) U.S. Cl. 435/32; 435/254.1; 435/255.1; 435/255.4; 435/256.1; 435/256.5; 435/256.3; 435/256.7

(57) ABSTRACT

The present invention provides a method of infecting a nail fragment with a fungus, to form an infected nail fragment comprising at least 1×102 fungal colony forming units (cfu) per cm2 of the infected surface(s). There is also provided a method of assessing the efficacy of potential therapeutic compounds in the treatment or prevention of a fungal nail infection. The fungal nail infection may be a dermatophyte, a non-dermatophyte mould or a pathogen.

### CROSS-REFERENCE TO RELATED APPLICATIONS

The present application is a U.S. National Phase of PCT application no. PCT/GB2010/002313 filed Dec. 20, 2010, which claims the benefit of GB application no. 0922403.1 filed on Dec. 22, 2009 and U.S. provisional application No. 61/289,911 filed on Dec. 23, 2009, each of which is incorporated herein by reference in its entirety for all purposes.

### FIELD OF THE INVENTION

The present invention relates to a method of establishing fungal nail infections ex vivo. There is also provided a method of testing the efficacy of compounds potentially effective in the treatment or prevention of fungal nail infections.

### BACKGROUND OF THE INVENTION

Fungal nail infections are common and account for approximately half of all nail abnormalities. It is believed that around 12% of the global population suffers from a fungal nail infection at any one time. Symptoms include the nail becoming thickened, yellow and/or cloudy. The surface of the nail commonly becomes rough and crumbly and the nail may separate from the nail bed. Fungal nail infections are unsightly and can be painful.

Onychomycosis (also known as tinea unguium) is a common fungal nail infection, affecting 6 to 8% of the global population at any one time.

Current treatments for fungal nail infections are relatively ineffective, some having an efficacy of only around 9%. In addition, current treatments for fungal nail infections involve repeated, regular applications resulting in much inconvenience and low compliance. This reduces the efficacy of current treatments still further.

Effective treatments for fungal nail infections are therefore sought. However, there is no accurate, reliable test to assess the effectiveness of potential treatments.

As nails are a specific feature of higher primate anatomy, there are no appropriate rodent or other animal models for the development of topical therapies for the treatment of fungal nail infections. There are obvious ethical concerns associated with the use of higher primates for the development of treatments for non-life threatening conditions, such as fungal nail infections, in addition to these models being costly and complex to undertake.

Currently, potentially therapeutic compositions for fungal nail infections are typically only tested through broth dilution antifungal susceptibility assays at the preclinical stage. These assays do not provide an accurate, reliable or predictive model of the activity of potential new treatments in vivo. A more physiologically relevant system is needed.

### SUMMARY OF THE INVENTION

According to a first aspect of the present invention there is provided a method of infecting a nail fragment with a fungus comprising the steps of:
a) obtaining a fungal sample comprising at least 1×10² fungal colony forming units per ml;
b) infecting a nail fragment with the fungal sample by applying the fungal sample to at least a surface of the nail fragment and incubating the nail fragment for an incubation period,
c) wherein the infected nail comprises at least 1×10² fungal colony forming units (cfu) per cm² of the infected surface(s).

Generally there is provided an ex vivo method of infecting a nail fragment and the nail fragment is removed prior to infection.

According to a further aspect of the present invention there is provided a method of assessing the efficacy of potentially therapeutic compounds in the treatment of a fungal nail infection comprising the step of:
a) identifying at least one potentially therapeutic compound,
b) obtaining a first nail fragment having a first surface, said first surface having a known number of fungal cfu per unit surface area, said known number being being at least 1×10² per cm²;
c) applying the potentially therapeutic compound to a surface of the nail fragment;
d) determining the number of fungal cfu per unit surface area of the first surface following application of the potentially therapeutic compound;
e) obtaining a second nail fragment, said second nail fragment having approximately the same thickness as the first nail portion wherein a first surface of the second nail fragment has approximately the same number of fungal cfu per unit surface area as the first surface of the first nail fragment;
f) applying a control composition to a surface of the second nail fragment;
g) assessing the number of fungal cfu per unit surface area of the first surface of the second nail fragment following application of the control composition;
h) comparing the number of fungal cfu per unit surface area of step d) and step g) wherein a number of fungal cfu per unit surface area of step d) lower than that of step g) is indicative of a compound effective in the treatment of the fungal nail infection.

The first and second nail fragments are preferably formed according to the method above.

According to a further aspect of the present invention there is provided a method of assessing the efficacy of potentially therapeutic compounds in the prevention of a fungal nail infection comprising the step of:
a) identifying at least one potentially therapeutic compound,
b) obtaining a first nail fragment having a first surface, said first nail fragment being uninfected with a fungal nail infection;
c) applying the potentially therapeutic compound to the first surface of the first nail fragment;
d) applying a fungal sample to a surface of the first nail fragment, said fungal sample comprising at least 1×102 fungal colony forming units per ml;
e) determining the number of fungal cfu per unit surface area of the first surface of the first nail fragment;
f) obtaining a second nail fragment, said second nail fragment having approximately the same thickness as the first nail fragment, wherein the second nail fragment is uninfected with a fungal nail infection;
g) applying a control composition to the first surface of the second nail fragment;
h) applying the fungal sample to a surface of the second nail fragment;
i) determining the number of fungal cfu per unit surface area of the first surface of the second nail fragment;
j) comparing the number of fungal cfu per unit surface area of step e) and step g) wherein a number of fungal cfu per unit surface area of step e) lower than that of step g) is indicative of a compound effective in the prevention of the fungal nail infection.

In contrast to known methods of testing compounds which are potentially therapeutic in the treatment or prevention of fungal nail infections, the methods of the present invention are accurate, repeatable, physiologically relevant and may be used in connection with all types of fungal nail infections. The environment used to test potentially therapeutic compounds according to the methods of the present invention is similar to the environment in which the compounds will be used. As such, the methods of the present invention provide a more accurate indication of how the potentially therapeutic compounds will behave in vivo. The use of a control composition maximises the accuracy of the method of the present invention. The method of the present invention provides an accurate ex vivo assay of how the potentially therapeutic composition will act in vivo.

### BRIEF DESCRIPTION OF THE DRAWINGS

The present invention will now be described by way of example only with reference to the accompanying Figures in which:

FIG. 1 illustrates the effect of compositions comprising 0.0%, 1.0%, 5.0% or 10.0% (w/v) 7 to 200-mer (SEQ ID NO: 3) polyarginine (Novexatin® (NP213)) with or without urea on the survival of T. rubrum on nail fragments treated daily for 28 days;

FIG. 2 illustrates the effect on T. rubrum of treatment with compositions comprising 0.0% (w/v), 2.5%, 5.0% or 10.0% (w/v) 7 to 200-mer (SEQ ID NO: 3) polyarginine (Novexatin® (NP213)), the compositions being applied daily or on alternate days;

FIG. 3 illustrates the effect on isolates of Trichophyton spp. on nail fragments treated with compositions comprising 50% (w/v) polyethylene glycol, 20% (w/v) urea and 0.0%, 5.0% or 10% (w/v) 7 to 200-mer (SEQ ID NO: 3) polyarginine (Novexatin® (NP213));

FIG. 4 illustrates the effect of simulated washing conditions on the survival of T. rubrum during treatment for 28 days of nail fragments with compositions comprising 50% (w/v) polyethylene glycol, 20% (w/v) urea and 0.0% or 10% (w/v) 7 to 200-mer (SEQ ID NO: 3) polyarginine (Novexatin® (NP213));

FIG. 5 illustrates the effect of treatment with compositions comprising 7 to 200-mer (SEQ ID NO: 3) polyarginine (Novexatin® (NP213)) and Fluconazole+/−urea on survival of T. rubrum during treatment of nail fragments for 14 days;

FIG. 6 illustrates the effect of 7 to 200-mer (SEQ ID NO: 3) polyarginine (Novexatin® (NP213)) and Fluconazole+/−urea on survival of T. rubrum during treatment of nail fragments for 28 days, the plates labelled C in FIG. 6 were contaminated with Penicillium spp. from an environmental source during the later stage of this experiment (day 22 to 24) and the values have been omitted;

FIG. 7 illustrates the effect of 7 to 200-mer (SEQ ID NO: 3) polyarginine (Novexatin® (NP213)) treatment on the survival of T. rubrum in infected human nail fragments, where FIG. 7a shows nail fragments treated with a control vehicle composition and FIG. 7b shows nail fragments treated with Novexatin® (NP213);

FIG. 8 illustrates the effect of 7 to 200-mer (SEQ ID NO: 3) polyarginine (Novexatin® (NP213)) treatment on the survival of Candida in infected human nail fragments compared to dRdRdRdRdRdRdRdRdRdRdRdRdR (SEQ ID NO: 2) (NP339), PEG/Urea, water, Itraconazole® in DMSO and DMSO;

FIG. 9 illustrates the effect of 7 to 200-mer (SEQ ID NO: 3) polyarginine (Novexatin® (NP213)) treatment on the survival of Candida in infected human nail fragments compared to PEG/Urea, dRdRdRdRdRdRdRdRdRdRdRdRdR (SEQ ID NO: 2) (NP339), water and Terbinafine®.

FIG. 10 illustrates the effect of 7 to 200-mer (SEQ ID NO: 3) polyarginine (Novexatin® (NP213)) on the prevention of of T. interdigitale at varying times from contact with T. interdigitale spore suspension, namely 30 minutes (FIG. 10a), 60 minutes (FIG. 10b), 24 hours (FIG. 10c), 7 days (FIG. 10d) and 14 days (FIG. 10e).

### DETAILED DESCRIPTION OF THE INVENTION

As used herein, the term “nail fragment” encompasses parts of a nail as well as complete nails.

A fungal sample may include one or more specified fungal cultures as well as pharmaceutically acceptable carriers or excipients. The mixture of fungal cultures in the fungal sample is generally known.

As used herein the term “compound” encompasses the pharmaceutically acceptable salts thereof.

The term “uninfected” is generally used to refer to less than 5 fungal colony forming units per ml, typically less than 1 fungal colony forming units per ml, suitably substantially zero fungal colony forming units per ml.

According to an aspect of the present invention there is provided a method of infecting a nail fragment with a fungus comprising the steps of:
obtaining a fungal sample comprising at least 1×10² fungal colony forming units (cfu) per ml;
infecting a nail fragment with the fungal sample by applying the fungal sample to at least a surface of the nail fragment and incubating the nail fragment for an incubation period,
wherein the infected nail comprises at least 1×10² fungal colony forming units (cfu) per cm² of the infected surface(s).

Potentially therapeutic compositions are generally tested through broth dilution antifungal susceptibility assays at the preclinical stage. Such assays do not provide an environment similar to that in which the compositions will be used. In contrast, the method of the present invention provides an accurate reliable test of the potentially therapeutic compositions in conditions similar to which they would be used. Surprisingly it has been found that some compounds exhibit far higher efficacy in the method of the present invention, and in vivo than would be expected from the results of broth dilution antifungal susceptibility assays. The method of the present invention thus provides a useful, accurate indication of how a potentially therapeutic composition is likely to behave in vivo, in particular when compared to current assays.

The potentially therapeutic composition may be potentially useful in the prevention and/or treatment of fungal nail infections.

The infected nail has generally been removed from the human or animal body prior to infection.

The fungus may be any which may cause a fungal nail infection. The fungus may be a dermatophyte, a non-dermatophyte mould or a pathogen.

The fungus is generally a dermatophyte, such as those isolated from a tinea infection, such as a tinea unguium, tinea corporis, tinea capitis, tinea cruris, tinea faciae and tinea pedis infection. The dermatophyte may be an isolate of Trichophyton spp. In particular the dermatophyte may be Trichophyton rubrum, Trichophyton mentagrophytes, Trichophyton violaceum, Trichophyton interdigitale, Trichophyton tonsurans, Trichophyton soudanense or Trichophyton verrucosum, Trichophyton schoenleinii, Epidermophyton floccosum, Microsporum gypseum, Microsporum audouinii or Microsporum canis.

Alternatively the fungus may be a yeast such as Candida spp, typically Candida albicans, Candida krusei, C. Glabrata, C. Famata, C. Parapsilosis, C. Tropicalis, C. Sake, Malassezia furfur and Trichosporon spp.

According to a further aspect of the present invention the fungus may be a non-dermatophyte mould such as Acremonium spp (for example A. roseogriseum), Alternaria spp., Arthrographis kalrae, Aspergillus spp. (including A. flavus, A. fumigatus, A. terreus, A. ustus, A. sydowii, A. versicolor), Arthroderma tuberculatum, Bipolaris spp., Botryodiplodia theobromae, Chrysosporium (Geomyces) pannorum, Cladosporium spp., Fusarium spp (including F. oxysporum, F. proliferatum, F. solani), Geotrichium candidum, Nattrassia spp., Onychocola canadensis, Paecilomyces spp., Penicillium spp., Phyllostricta sydowii, Pyrenochaeta unguis-hominis, Scopulariopsis brevicaulis, Scytalidium spp. (including S. didmidiatum, S. hyalinum), Synchephalastrum racemosum, Trichoderma spp. and Ulocladium spp.

The fungal sample may comprise a single fungal culture, or a mixture of more than one fungal culture. The identity of the fungal cultures contained in the fungal sample would generally be known prior to application. According to one embodiment, the fungal sample may comprise more than one fungal culture, typically two or three fungal cultures.

According to one embodiment, the fungal sample may comprise more than one dermatophyte. The fungal sample may comprise at least one dermatophyte and one or more non-dermatophyte mould or more than one yeast.

According to one embodiment the fungal sample may comprise more than one yeast.

Generally the fungal sample comprises at least 1×10² fungal colony forming units per ml, typically at least 1×10³, suitably at least 1×10⁴ , more suitably 5×10⁴ fungal cfu per ml.

The fungal sample typically comprises one or more pharmaceutically acceptable excipients or carriers such as solvents, anti-adherants, binders, fillers, diluents, lubricants and preservatives.

Typically the fungal sample comprises less than 1% by volume impurities; generally less than 0.5% by volume impurities, suitably less than 0.1% by volume impurities.

The step of infecting the nail may involve applying the fungal sample to at least the ventral side of the nail, where the ventral side of the nail is the side attached to the body prior to removal, and the dorsal side of the nail is the side facing away from the body, towards the environment prior to removal. Typically the fungal sample is only applied to the ventral side of the nail. According to one embodiment of the invention the infected nail has a ventral side comprising at least 1×10² fungal colony forming units (cfu) per cm², and a dorsal side comprising less than 5 fungal cfu per cm², typically approximately zero fungal cfu per cm². The infected nail thus provides an ex vivo model of an in vivo nail infected with a fungal nail infection, especially subungual onychomychosis or Candida.

The fungal sample may be applied to the nail fragment using any suitable technique, for example the fungal sample may be applied by painting, spreading or spraying the fungal sample onto the nail fragment. Alternatively, the nail fragment may be dipped into the fungal sample.

The duration and conditions applicable for the incubation period are dependent on many variables including the number of fungal cfu in the fungal sample, the fungal culture(s) in the fungal sample, the thickness of the nail fragment and the side of the nail to which the fungal is applied to. The incubation period is also dependent on the desired number of cfu on the infected nail fragment. The incubation period is sufficient to allow a fungal infection to become established on the nail fragment and this may be determined at least in part through inspection of the nail fragment, including visual inspection. Visual symptoms of a fungal nail infection include discolouration, increased nail weakness and the presence of fungal hyphae on the surface of the nail.

The incubation period is typically up to 5 to 70 days, generally up to 50 days, suitably 20 days or less, more suitably up to 14 days. Where the fungal sample is applied to the ventral surface of the nail fragment, the incubation period is typically 20 days or less, generally 7 to 14 days. Where the fungal sample is applied to the dorsal surface of the nail fragment, the incubation period may be significantly longer, typically 30 to 70 days.

The nail fragment may be incubated at temperatures elevated from room temperature following application of the fungal sample. The elevated temperature may be 25 to 40 degrees Celsius, generally 25 to 40 degrees Celsius, suitably 30 to 37 degrees Celsius, more suitably around 30 degrees Celsius+/−1 degree Celsius.

Typically the incubation period is conducted under elevated humidity levels. Suitably the humidity levels are greater than 40%, typically greater than 50%.

The infected nail comprises at least 1×10² fungal colony forming units (cfu) per cm² of the infected surface(s), typically 1×10³, suitably 1×10⁴, more suitably 5×10⁴. According to one embodiment, the ventral side of the infected nail comprises at least 1×10² fungal cfu per cm², and the dorsal side of the infected nail comprises less than 5 cfu per cm², typically less than 1 cfu per cm².

Visual analysis may provide an indication that the nail is infected. Typical visual signs include discolouration, increased nail weakness and the presence of fungal hyphae on the surface of the nail.

The infected nail is typically infected with onychomycosis. Alternatively the infected nail may be infected with Candida.

Typically the fungal sample is a cultured fungal sample obtainable according to the steps of:
obtaining a fungal isolate;
growing the fungal isolate generally for 1 to 14 days at 30 to 37 degrees Celsius, typically on an appropriate sterile agar such as Sabouraud dextrose agar or potato dextrose agar until the fungal isolate has grown substantially, generally to cover the entire surface of the agar and has produced aerial hyphae where appropriate,
and subsequently following the instructions for preparation of a standard inoculum described in the Clinical and Laboratory Standards Institute (CLSI) Approved Standard (M38-A2); Reference Method for Broth Dilution Antifungal Susceptibility Testing of Filamentous Fungi (2^{nd} Edition) or CLSI) Approved Standard (M27-A3); Reference Method for Broth Dilution Antifungal Susceptibility Testing of Yeasts (3^{rd} Edition).

Typically, an innoculum may be prepared by forming a culture stock by combining the fungal isolate with a cryoprotectant, such as dimethyl sulfoxide (DMSO) at typically 1 to 5% v/v in Sabouraud Dextrose broth. Aliquots (typically 50 microlitres) of the culture stock may be transferred to slopes of sterile Agar medium, normally Potato Dextrose Agar or Oatmeal Agar and incubated at elevated temperature, typically more than 25 degrees Celsius, suitably 28 to 35 degrees Celsius, more suitably approximately 30 degrees Celsius. Incubation is typically for up to 7 days, suitably 5 to 7 days or until the isolate forms a clearly visible mat on the surface of the agar. A spore suspension is prepared by adding water (typically around 3 millilitres) to the culture tube and and filtering the resultant solution.

The nail is generally a fingernail or toenail obtained from a mammal, generally a human. Alternatively the nail may be obtained from a primate. Advantageously the nail is not infected with any fungal nail infection prior to the method of the present invention.

According to a further aspect of the present invention there is provided a method of assessing the efficacy of potential therapeutic compounds in the treatment of a fungal nail infection comprising the step of:
a) identifying at least one potentially therapeutic compound,
b) obtaining a first nail fragment having a first surface, said first surface having a known number of fungal cfu per unit surface area being at least 1×10² per cm²;
c) applying the potentially therapeutic compound to a surface of the nail fragment;
d) assessing the number of fungal cfu per unit surface area of the first surface following application of the potentially therapeutic compound;
e) obtaining a second nail portion, said second nail portion having approximately the same thickness as the first nail portion wherein a first surface of the second nail fragment has approximately the same number of fungal cfu per unit surface area as the first surface of the first nail portion;
f) applying a control composition to a surface of the second nail fragment;
g) assessing the number of fungal cfu per unit surface area of the first surface of the second nail portion following application of the control composition;
h) comparing the number of fungal cfu per unit surface area of step d) and step g) wherein a number of fungal cfu per unit surface area of step d) lower than that of step g) is indicative of a compound effective in the treatment of the fungal nail infection.

The infected nail portion is typically removed prior to the method of the present invention. Generally the infected nail is prepared according to the method described above.

The surface to which the potentially therapeutic compound is applied may be the first surface or a surface other than the first surface. The potentially therapeutic compound and the control composition are applied to the same surface of the first and second nail portions.

Typically the first surface is the ventral side of the nail. Suitably the potentially therapeutic compound and control composition are applied to the dorsal side of the nail. To be effective in reducing the number of cfu on, and in the nail matrix of the nail, a therapeutic composition would have to penetrate the dorsal surface, infiltrate through the nail matrix and reach the ventral side of the nail. Thus, the method of the present invention provides an accurate in vitro model of in vivo infected nails, in particular those infected with subungual onychomychosis infection, or those infected with Candida.

According to one embodiment, steps b) to g) may be repeated before step h) is performed. Steps b) to g) are generally repeated the same number of times. Typically steps b) to g) are repeated at least ten times before step h) is performed. Suitably, steps b) to g) are performed daily for at least fourteen days before step h) is performed; generally daily for at least twenty eight days.

Alternatively, steps b) to g) may be performed on alternate days for at least fourteen days, typically twenty-eight days.

A washing procedure may be introduced between applications of the potentially therapeutic compound and control composition. Surprisingly it has been found that such a washing procedure improves the efficacy of some potentially therapeutic compounds. It has been found that in some instances, the introduction of a washing procedure decreases the efficacy of the control composition.

Typically a cfu per unit surface area of step d) at least 5% less than that of step g) is indicative of a compound effective in the treatment of the fungal nail infection; suitably 10% or less, more suitably 20% or less, generally at least 50% less, preferably at least 75% less, more preferably at least 90% less, advantageously at least 99% less.

According to one embodiment, following application of the potentially therapeutic compound to the nail the number of cfu is so low that the infection cannot re-establish itself.

Generally the first and second nail fragments are produced according to the method detailed above.

According to a further aspect of the present invention there is provided a method of assessing the efficacy of potentially therapeutic compounds in the prevention of a fungal nail infection comprising the step of:
a) identifying at least one potentially therapeutic compound,
b) obtaining a first nail fragment having a first surface, said first nail fragment being uninfected with a fungal nail infection;
c) applying the potentially therapeutic compound to the first surface of the first nail fragment;
d) applying a fungal sample to a surface of the first nail fragment, said fungal sample comprising at least 1×10² fungal colony forming units per ml;
e) determining the number of fungal cfu per unit surface area of the first surface of the first nail fragment;
f) obtaining a second nail fragment, said second nail fragment having approximately the same thickness as the first nail fragment, wherein the second nail fragment is uninfected with a fungal nail infection;
g) applying a control composition to the first surface of the second nail fragment;
h) applying the fungal sample to a surface of the second nail fragment, said fungal sample comprising at least 1×10² fungal colony forming units per ml;
i) determining the number of fungal cfu per unit surface area of the first surface of the second nail fragment;
j) comparing the number of fungal cfu per unit surface area of step e) and step g) wherein a number of fungal cfu per unit surface area of step e) lower than that of step g) is indicative of a compound effective in the prevention of the fungal nail infection.

Typically, the fungal sample is applied to a surface other than the first surface.

Generally the first surface of the nail fragments is the dorsal side of the nail. Generally, the potentially therapeutic compound and control composition are applied to the dorsal side of the nail, and the fungal sample is applied to the ventral side of the nail.

Generally step c) and step g) are repeated the same number of times, typically 1 to 14 times, suitably 1 to 10 times. According to one embodiment the potentially therapeutic compound and the control compound of step c) and step g) are repeated once a day for 1 to 7 days.

According to one embodiment, steps b) to h) may be repeated before step i) is performed. Steps b) to h) are generally repeated the same number of times. Typically steps b) to h) are repeated at least ten times before step i) is performed. Suitably, steps b) to h) are performed daily for at least twenty days before step h) is performed; generally daily for at least fourteen days, typically for one to seven days.

According to one embodiment of the present invention, the thickness of the first and second nail portions are the same to within 10%, typically 5%, suitably 1%, advantageously 0.1%.

Generally the area of the first and second nail portions is the same to within 10%, typically 5%, suitably 1%, advantageously 0.1%.

Typically the cfu per unit area of the first and second nail portions is the same to within 10%, typically 5%, suitably 1%, advantageously 0.1%.

The nail fragment may be a portion of an excised nail. Alternatively, the nail fragment may be the whole of an excised nail.

Typically a cfu per unit surface area of step e) at least 5% less than that of step g) is indicative of a compound effective in the prevention of the fungal nail infection; suitably 10% or less, more suitably 20% or less, generally at least 50% less, preferably at least 75% less, more preferably at least 90% less, advantageously at least 99% less.

Typically the potentially therapeutic compound is applied to the nail fragment in the form of a composition. The composition typically comprises the potentially therapeutic compound and one or more pharmaceutical excipients.

The potentially active compound may be any compound of interest in the treatment or prevention of a fungal nail infection. According to one embodiment, the potentially active compound may be a peptide, small molecule, allylamine (such as amorolfine, butenafine, naftifine and terbinafine), azole (such as abafungin, bifonazole, butaconazole, clotrimazole, econazole, fenticonazole, fluconazole, isaconazole, isavuconazole, itraconazole, ketoconazole, miconazole, oxiconazole, posaconazole, ravuconazole, serticonazole, sulconazole, terconazole, tioconazole and voriconazole), polyene (such as amphotericin B, candicin, filipin, hamycin, natamycin, nystatin and rimocidin), echinocandin (such as anidulafungin, caspofungin and micafungin), benzoic acid, ciclopirox olamine, flucytosine, griseofulvin, haloprogin, piroctone olamine, selenium sulphide, sodium bicarbonate, tolnaftate, undecylenic acid, zinc pyrithione or other alternative therapies. Additionally, different formulations of potentially active compounds can be used in this model.

The control composition may consist of the pharmaceutical excipients of the potentially therapeutic composition. Typically, the control composition consists of the same components as the potentially therapeutic composition, absent the potentially therapeutic compound.

Suitable pharmaceutical excipients include anti-adherents, binders, coatings, disintegrants, fillers and diluents, flavours, colours, glidants, lubricants, preservatives, sorbents, solvents, stabilisers, and sweeteners, such as polyethylene glycol and urea, and as described in the US Food and Drug Administration “Inactive Ingredient Approved Drug Products” database.

Generally approximately the same volume of potentially therapeutic composition and control composition per unit area are applied to the first and second nail fragments. Typically the volume per unit area is the same to 5% or less, suitably 2% or less; more suitably 1% or less; advantageously 0.1% or less. According to one embodiment, 5 to 15 microlitres/cm² of potentially therapeutic composition and control composition are applied to first and second nail fragments respectively. Typically approximately 10 microlitres/cm² is applied. Generally the potentially therapeutic compound and the control composition are applied using the same method, for example, paiting, spraying, dipping etc.

The concentration of potentially therapeutic compound in the potentially therapeutic composition is dependent on the fungal nail infection to be treated. The concentration is typically 1 to 5% w/v, suitably 3% w/v or less. Alternatively, the concentration may be 20% w/v or less, generally 5 to 15% w/v, typically 5 to 10% w/v. The concentration may be 1, 5 or 10% w/v.

At least 10× the Minimum Inhibitory Concentration (MIC) of the potentially therapeutic compound may be applied to the nail fragment, typically 20× the MIC, suitably 50× the MIC. Advantageously, at least 100× the MIC of the potentially active compound is applied to the nail fragment.

The reduction of the number of fungal cfu after application of the control composition is generally 5% or less, suitably 2% or less, typically less than 0.5%. According to one embodiment, there is no reduction in the number of fungal cfu above the level of detection of the assay.

The method may include the step of macroscopic visual inspection of the first and second nail portions to assess the degree of fungal nail infection. Generally a nail suffering from a fungal nail will appear visually infected at a macroscopic level. Typical visual signs of infection include a thickening of the nail, discoloration and flakiness.

The therapeutic compositions tested are generally potentially effective against any fungal nail infection. Examples of typical fungal nail infections include distal subungual onychomycosis, white superficial onychomycosis, proximal subungual onychomycosis and/or candidal onychomycosis.

According to one aspect of the present invention, the method of assessing efficacy may be repeated using a different potential therapeutic compound. The number of fungal cfu per unit surface area following application of the second potential therapeutic compound may be assessed. This may be compared with the number of fungal cfu per unit surface area following application of the control and/or the number of fungal cfu per unit surface area following application of the first potential therapeutic compound.

### EXAMPLES

### 1. Materials and Methods

1.1 Preparation of Test Item and Vehicle

Ten vials of the acetate salt of 7 to 200-mer (SEQ ID NO: 3) polyarginine (Novexatin® (NP213)), each of 1 g (net) (NeoMPS SA, Strasbourg France, Batch No. ED02098), were received by NovaBiotics on 6 Apr. 2007. The test item is a neutral, white, amorphous powder with a peptide content of 67.5% and purity of 98.4%, and was stored at −80° C. in the dark following receipt.

For antifungal efficacy studies with human toe nails, a solution of 50% (w/v) PEG 8000 (Sigma-Aldrich; Cat No P2139) was prepared by autoclaving and then diluting with sterile 50% (w/v) urea solution (Sigma-Aldrich; Cat No 51457). This solution was mixed thoroughly by vortexing and allowed to stabilise at 50° C. for up to 2 h. After stabilisation, the temperature was allowed to drop to below 40° C. and a concentrated, filter-sterilised solution of Novexatin® (NP213), dissolved in deionised water was added. Final concentrations of PEG 8000 of 50% (w/v), urea of 0-20% (w/v) and Novexatin® (NP213) of 0-10% (w/v) were used in the experiments described below.

Sterile pipette tips and other plastic-ware used in the process were pre-warmed to 50° C. prior to use. Solutions containing high concentrations of Novexatin® (NP213) occasionally formed an opaque, semi-solid mass on refrigerated storage but rapidly became a clear, viscous liquid on warming at 40° C.

1.2 Preparation of Test Organisms for Ex vivo Antifungal Efficacy Studies

Trichophyton rubrum strain NCPF0118 was obtained from the National Collection of Pathogenic Fungi, Bristol, UK. Clinical isolates of T. rubrum DM2006 1661, T. rubrum DM2006 1358, T. mentagrophytes DM2006 1503, T. mentagrophytes DM2006 1498 were isolated from patients suffering a range of tinea infections and were purchased from Professor Michel Monod (Service de Dermatologie, Université de Lausanne, Switzerland). Clinical isolates of Candida samples were also obtained from patients suffering from Candidal infection.

On arrival, the cultures were transferred to a Petri dish containing sterile Sabouraud Dextrose agar (Sigma-Aldrich; Cat No 84088, or equivalent) and grown for up to 7 d at 30° C. The resulting growth was scraped off and re-suspended in Sabouraud's liquid medium (Oxoid; Cat No CM 147, or equivalent) containing 0.5% (v/v) dimethyl sulfoxide (DMSO) as cryoprotectant to create a culture stock. The fungus was stored in 0.5 ml aliquots in 2 ml screw-capped polypropylene vials (Cryovials) at −80° C. Seven days prior to experimental use, 50 μl of the culture stock was used to inoculate a slope of Potato Dextrose agar (Merck; Cat No VM556130 609, or equivalent), which was incubated at 30° C. for up to 7 d. The surface of the resulting growth was gently perturbed in 3 ml of sterile distilled/deionised water. After filtration through 2 layers of sterile surgical gauze to remove debris and larger clumps of biomass, the absorbance of the resultant spore suspension was measured at 530 nm, then adjusted to the 0.5 MacFarland standard (OD approx. 0.15)².

1.3 Human Nail Fragments

Human toe nails were obtained from a podiatrist in Aberdeen following a successful application to the Grampian Local Research Ethics Committee. The nails chosen for study were not discoloured and showed no sign of disease or infection. Large toe nails were cut into 4 pieces, each approximately 1 cm² in area, using nail clippers. Nails of normal thickness (≦2 mm) were chosen. The 4 fragments cut from each single nail were placed in quadrants marked on Petri-dishes containing phosphate buffered saline rendered solid with 1.2% (w/v) Technical Agar No. 3 (Oxoid; Cat No LP0013, or equivalent), subsequently referred to as PBS agar. The nail fragments were of irregular shape, so to ensure that each nail received the appropriate amount of the treatment solutions, the surface area was measured by photographing the nails over graph paper and determining the number of 1mm² squares covered by each nail. The thickness of the nails was measured with a micrometer and nails distributed among treatment groups, to avoid nail thickness providing an additional experimental variable.

1.4 Inoculation and Treatment of Nails

In total, descriptions of seven variations on a common experimental design are reported here. The efficacy of the following agents against fungal infection were tested using the ex vivo nail model of the present invention:
7 to 200-mer (SEQ ID NO: 3) polyarginine (Novexatin® (NP213)), generally 7-mer cyclic polyarginine (NP213) was used.
dRdRdRdRdRdRdRdRdRdRdRdRdR (SEQ ID NO: 2) (NP339)
(2R,4S)-rel-1-(butan-2-yl)-4-{4-[4-(4-{[(2R,4S)-2-(2,4-dichlorophenyl)-2-(1H-1,2,4-triazol-1-ylmethyl)-1,3-dioxolan-4-yl]methoxy}phenyl)piperazin-1-yl]phenyl}-4,5-dihydro-1H-1,2,4-triazol-5-one (Itraconazole®)
[(2E)-6,6-dimethylhept-2-en-4-yn-1-yl](methyl)(naphthalen-1-ylmethyl)amine (Terbinafine®)
2-(2,4-difluorophenyl)-1,3-bis(1H-1,2,4-triazol-1-yl)propan-2-ol (Fluconazole)

1.4.1 Experiment 1

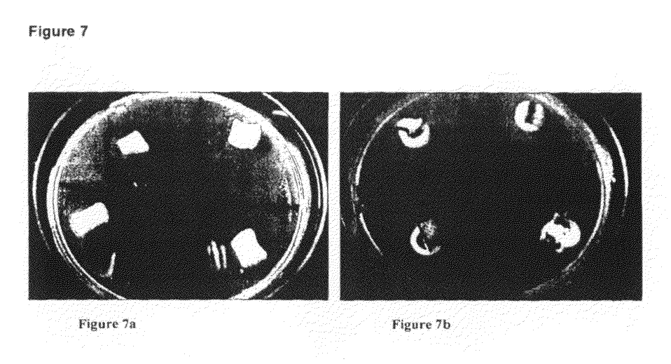

Twenty four nail fragments from 6 whole large toenails were each inoculated with 10 μl of a spore suspension of T. rubrum NCPF0118 which was prepared as described above. The inoculum was applied to the ventral side of the nail (i.e. the side next to the skin in situ). The nails were incubated for 7 d at 30° C. to allow the fungus to infect the nail tissue. After this pre-incubation period, 12 of the fragments were assigned to each of 2 different treatment protocols, involving daily treatment with 2 different vehicle solutions, each containing a control and 3 different concentrations of Novexatin® (NP213). These vehicle solutions were: 50% (w/v) PEG 8000, and 50% (w/v) PEG 8000 plus 20% (w/v) urea. The concentrations of Novexatin® (NP213) studied were 0%, 1%, 5% and 10% (w/v). The solutions were applied at a volume of 10 μl/cm², corrected for the surface area of the different nail fragments as described above. The applied solution was spread over the dorsal side of the nail, to mimic the intended clinical treatment and the ventral side of the nail rested on the agar surface. Each time the nails were treated, they were transferred to a new sterile Petri dish containing PBS agar to minimise any build-up of fungal growth on the agar. Nail fragments were treated daily for 28 days. Triplicate colony counts were conducted in connection with each nail fragment, and a visual examination of the nail fragments was conducted (see FIG. 7).

1.4.2 Experiment 2

The procedure was carried out as described above but the vehicle solution contained 50% (w/v) PEG 8000 and 20% (w/v) urea, with Novexatin® (NP213) at final concentrations of 0%, 2.5%, 5% and 10% (w/v). Nails were treated daily, or on alternate days to mimic non-compliance with the intended treatment protocol.

1.4.3 Experiment 3

The experiment was performed as described in Experiment 1 with modifications relating to the vehicle, concentrations and fungal strains used. The vehicle used was a 50% (w/v) PEG 8000 plus 20% (w/v) urea and the concentrations of Novexatin® (NP213) studied were 0%, 5% and 10% (w/v). The fungal strains tested were clinical isolates of T. rubrum DM 2006 1661, T. rubrum DM 2006 1358, T. mentagrophytes DM 2006 1503, T. mentagrophytes DM 2006 1498, which were isolated from tinea unguium, tinea unguium, tinea faciae and tinea pedis infections, respectively. In addition, restrictions on the availability of nail material dictated that each treatment was applied in duplicate, compared with Experiment 1, where nails were treated in triplicate. Triplicate colony counts were conducted in connection with each nail fragment.

1.4.4 Experiment 4

The experiment was performed as described in Experiment 1 (with the omission of the 1% (w/v) Novexatin® (NP213) treatment) but immediately prior to application of Novexatin® (NP213) the upper surface of the nails was “washed” with sterile water using a sterile cotton bud to simulate washing/bathing. It is anticipated that users of the compound will be instructed to wash the affected area before applying the compound, and not to wash immediately afterwards. It was important to know if removal of excess Novexatin® (NP213) by later washing would diminish the effectiveness of the treatment. The nails were incubated in agar plates as above, but were held above the agar surface by resting on sterile polypropylene rings (See FIG. 7) to allow access of oxygen to the fungi growing on the ventral nail surface and to facilitate treatments (including washing) that required the nails to be picked up using sterile forceps for handling.

1.4.5 Experiment 5

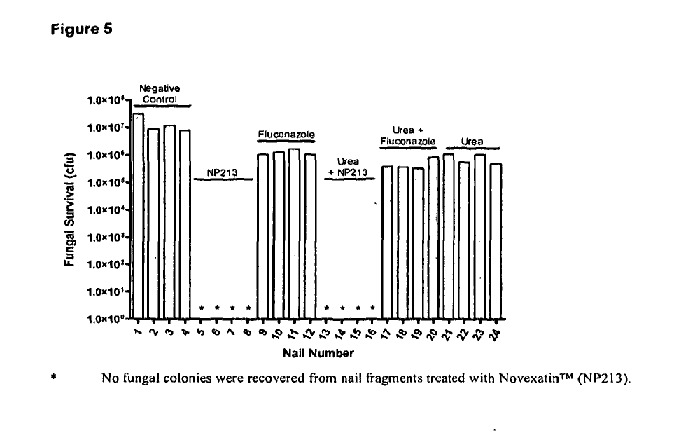

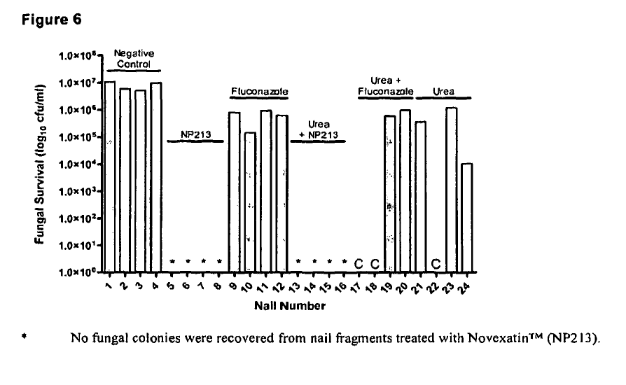

This experiment was performed as described in Experiment 4, but used fluconazole as a comparator water-soluble antifungal agent. Other antifungal agents were not tested as they require an organic solvent to attain dissolution at experimentally relevant concentrations. Organic solvents are known to interfere with fungal growth and would therefore lead to the generation of artefactual results. The highest dose of Novexatin® (NP213) used was 10% (w/v), which is approximately 100× the MIC₁₀₀ versus T. rubrum NCPF0118 (revealed in separate tests). Using the methods described for experiments 1 and 2 above, a comparison was made of the effectiveness of Novexatin® (NP213) versus fluconazole. For the purpose of this comparison, fluconazole was used at 100× the MIC₁₀₀ (0.36 mg/ml) versus T. rubrum NCPF0118 in vitro. Fluconazole was prepared as an 8 mg/ml solution in sterile, deionised water. Nail fragments were treated daily. Each nail fragment was assessed at 14 days (FIG. 5) and 28 days (FIG. 6). Fungal cfu counts were determined from triplicate colony counts from each nail fragment.

1.4.6 Experiment 6

The procedure was carried out as described for Example 1 above with modifications to the vehicle, and fungal strains used. Each nail was inoculated with 10 μl of a spore suspension of Candida. Following inoculation the nails were treated with 10% (w/v) Novexatin® (NP213) in a vehicle solution of 50% (w/v) PEG 8000 plus 20% (w/v) urea; 10% (w/v) NP339 in a vehicle solution of 50% (w/v) PEG 8000 plus 20% (w/v) urea; 10% (w/v) NP339 in a vehicle solution of water; a control solution of 50% (w/v) PEG 8000 plus 20% (w/v) urea; a control solution of water; a solution of 16 μg/ml Itraconazole® and 1.6% (w/v) DMSO in a vehicle solution of water and a control solution of 1.6% (w/v) DMSO. As for Experiment 1 above, nail fragments were treated daily for up to 28 days.

1.4.7 Experiment 7

The procedure was carried out as described for Example 6 above except that the nails were treated with a solution of 10 μg/ml Terbinafine® in rather than the Itraconazole® solution.

1.4.8 Experiment 8

Six nail fragments were obtained. Three were assigned to each of two different infection prevention protocols, involving daily application of 2 different vehicle solutions, each containing a control and 3 different concentrations of Novexatin® (NP213). These vehicle solutions were: 50% (w/v) PEG 8000, and 50% (w/v) PEG 8000 plus 20% (w/v) urea. The concentrations of Novexatin® (NP213) studied were 0% (control), 1% (Novexatin® 1), 5% (Novexatin® 2) and 10% (Novexatin® 3) (w/v). The solutions were applied at a volume of 10 μl/cm², corrected for the surface area of the different nail fragments as described above. The applied solution was spread over the dorsal side of the nail, to mimic the intended clinical treatment and the ventral side of the nail rested on the agar surface. Each time the nails were contacted with the vehicle solutions, they were transferred to a new sterile Petri dish containing PBS agar to minimise any build-up of fungal growth on the agar. Nail fragments were contacted with the vehicle solutions daily for 7 days. Following such contact, each nail fragment was contacted with 10 μl of a spore suspension of T. interdigitale NCPF0335 which was prepared as described above. The spore suspension was applied to the ventral side of the nail (i.e. the side next to the skin in situ). The growth of T. interdigitale on each of the nail fragments was tested at 30 minutes, 60 minutes, 24 hours, 7 days and 14 days from contact of the nail fragments with the spore suspension.

1.5 Assessment of Fungal Growth

At the end of the treatment period, the nail fragments were placed in sterile 2 ml microcentrifuge tubes containing 450 μl of a suspension broth consisting of Sabouraud's Liquid Medium, pH 5.6, containing approximately 100 mg of sterile zirconia beads (0.5 mm diameter). From Experiment 4 onwards, 5% (w/v) polyanethole sulphonic acid was added to the suspension broth. The samples were vortex-mixed to release fungi from the nails and a dilution series was prepared from 10⁰ to 10⁻⁵. Triplicate aliquots of 10 μl of each dilution were spread on plates of Sabouraud Dextrose Agar and incubated for up to 5 d at 30° C. to allow colonies to develop. Colonies were counted on dishes that contained between 20 and 200 colonies in total, and the total number of cfu present in each sample was calculated. If 2 or more dilutions fell within the countable range (20-200 colonies per plate), or fewer than 20 colonies were present on a plate and a higher dilution was available, the dilution containing the larger number of colonies was selected. The counts were corrected for the surface area of the nails studied to give cfu/cm², transformed to log₁₀ and means and standard errors of the mean were calculated using GraphPad Prism 4 (GraphPad, San Diego, Calif.). Statistical analysis by two-way Anova was also performed using GraphPad Prism 4. The lowest number of counts which could be enumerated accurately corresponded to approximately 10³ cfu/cm². When very low numbers of colonies were detected, as in Experiment 3, the calculated cfu values are shown in order to indicate trends.

### 2. Results

2.1 Survival of Trichophyton rubrum NCPF0118 and Candida on Human Nail Fragments

2.1.1 Experiment 1

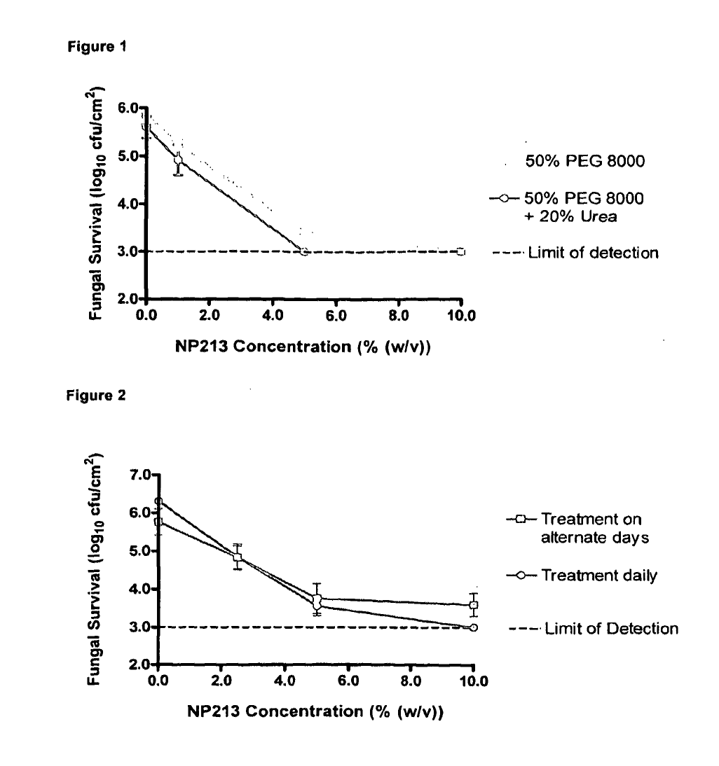

As can be seen from FIG. 1, Novexatin® (NP213) kills T. rubrum NCPF0118 in a dose-dependent manner. This was also apparent from fungal counts, and from the appearance of the Novexatin®-treated nails, as illustrated for later experiments in FIG. 7. When nail fragments were treated with 10% (w/v) Novexatin® (NP213), no colonies were isolated in both the presence and absence of urea. When nail fragments were treated with 5% (w/v) Novexatin® (NP213) with 20% (w/v) urea in the vehicle no colonies were isolated. However, a small number of colonies were detected in the absence of urea and in the presence of 5% (w/v) Novexatin® (NP213), although this treatment still represents an approximately 3 log₁₀ (1,000-fold) decrease in survival of the fungus compared to the controls which were not treated with Novexatin® (NP213). Treatment with 1% (w/v) Novexatin® (NP213) resulted in an approximately 1 log₁₀ (10-fold) reduction in fungal survival. Analysis by two-way Anova showed that the effect of treatment with 5% or 10% (w/v) Novexatin® (NP213) was highly significant (p<0.0001) but that the numbers of cfu recovered after treatment with 5% (w/v) Novexatin® (NP213) in vehicle with or without urea did not differ significantly. Overall, the numbers of cfu recovered were lower when urea was present in the vehicle.

2.1.2 Experiment 2

The purpose of Experiment 2 was to determine whether treatment of infected nail fragments with Novexatin® (NP213) had to be carried out on a daily basis, or whether treatment could be carried out on alternate days. Based on the results from Experiment 1, the vehicle included 20% (w/v) urea and 2.5% (w/v) Novexatin® (NP213) was substituted for the minimally effective 1.0% (w/v) Novexatin® (NP213) treatment. As can be seen in FIG. 2, there was a dose-dependent effect of Novexatin® (NP213) treatment on fungal survival. When 10% (w/v) Novexatin® (NP213) was applied on a daily basis, no fungal colonies were recovered but when the treatment was applied on alternate days, small numbers of colonies were recovered. This treatment still represents approximately a 3 log₁₀ (1,000-fold) decrease in survival of the fungus, compared to the controls which were not treated with Novexatin® (NP213). Analysis of the results by two-way Anova confirmed that the effect of Novexatin® (NP213) was statistically significant (p<0.0001) and that the differences in results between daily and alternate-day treatment were not significantly different. In this experiment, treatment with 5% (w/v) Novexatin® (NP213) was less effective in reducing fungal survival than observed in Experiment 1, although the difference was not statistically significant.

2.1.3 Experiment 3

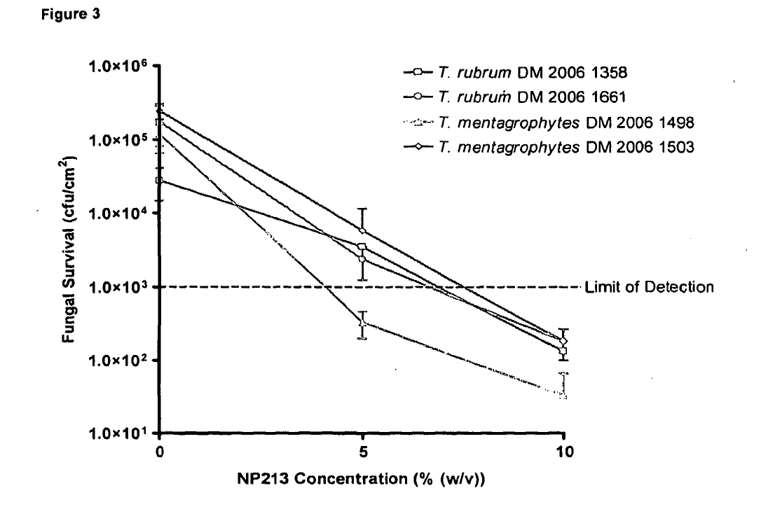

The purpose of Experiment 3 was to determine the antifungal efficacy of Novexatin® (NP213) against a random selection of clinical isolates of Trichophyton spp. isolated from tinea infections, including tinea unguium (onychomycosis). As can be seen from FIG. 3, survival of these 4 strains was similar to that recorded for T. rubrum NCPF0118 (shown in FIGS. 1 & 2). Again, the effect of Novexatin® (NP213) was dose-dependent.

2.1.4 Experiment 4

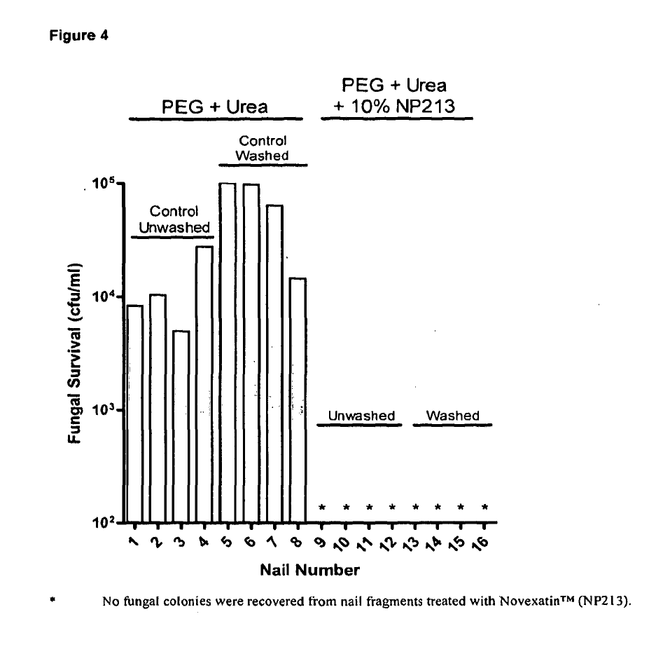

The purpose of Experiment 4 was to determine the antifungal efficacy of Novexatin® (NP213) during simulated “washing” of nail fragments to simulate washing of feet during treatment with Novexatin® (NP213). The data from this experiment showed that wiping the surface of the nails with cotton buds soaked in sterile water did not reduce the effectiveness of recovery of T. rubrum NCPF0118 from untreated nails, and did not affect the antifungal efficacy of Novexatin® (NP213) (FIG. 4). No fungal colonies were recovered from washed or unwashed nail fragments treated with Novexatin® (NP213). Perhaps surprisingly, the washing process tended to increase the recovery of the test strain from the untreated control nail fragments (FIG. 4).

2.1.5 Experiment 5

The purpose of Experiment 5 was to compare directly the antifungal efficacy of Novexatin® (NP213) and fluconazole (a clinically relevant antifungal agent, effective against dermatophytes) in the ex vivo nail model system. When Novexatin® (NP213) and fluconazole were applied to the dorsal surface of infected nails at concentrations approximately 100×MIC₁₀₀ against T. rubrum NCPF0118, treatment with Novexatin® (NP213) for either 14 d or 28 d resulted in no recoverable fungal growth at both time points (FIGS. 5 and 6). However, treatment with fluconazole was unsuccessful after both 14 d and 28 d of treatment.

2.1.6 Experiment 6

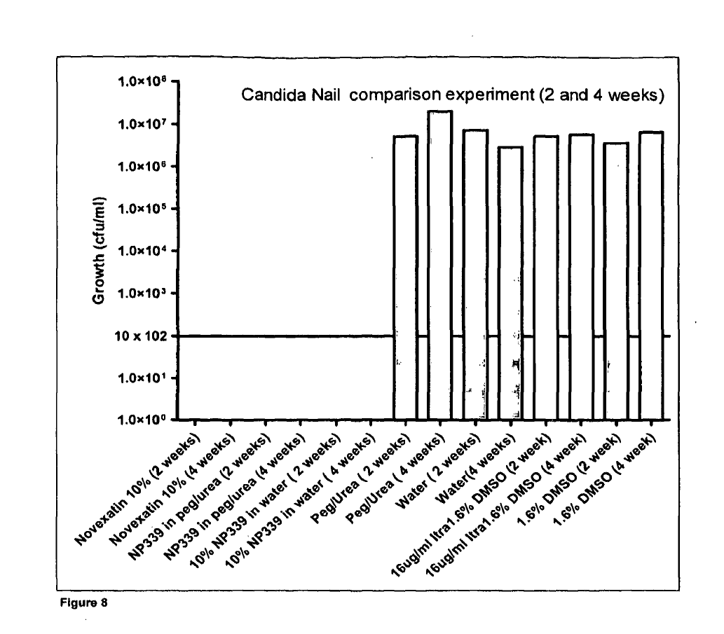

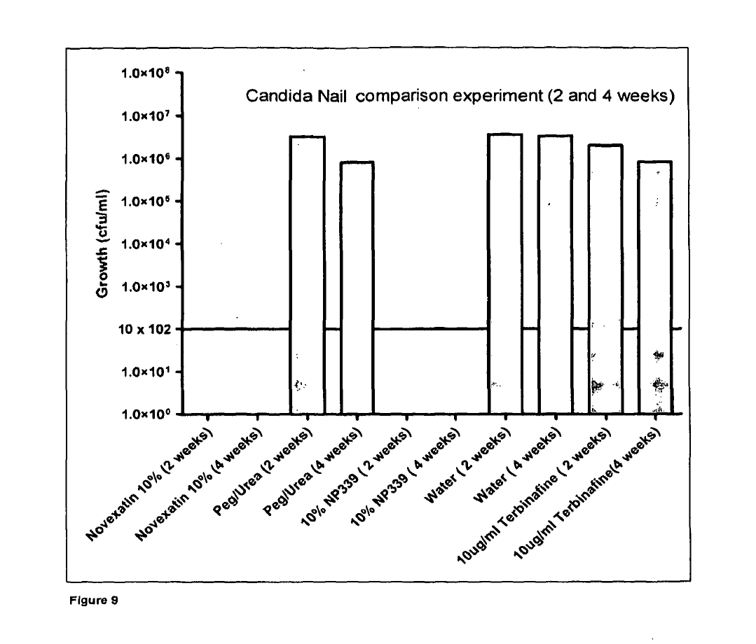

The purpose of Experiment 6 was to compare directly the antifungal efficacy of Novexatin® (NP213), NP339 and Itraconazole® (a marketed antifungal agent) in the ex vivo nail system. When Novexatin® (NP213), NP339 and Itraconazole® were applied to the dorsal surface of infected nails treatment with Novexatin® (NP213) and NP339 for either 14 d or 28 d resulted in no recoverable fungal growth at both time points (FIG. 8). However, treatment with Itraconazole® was unsuccessful after both 14 d and 28 d of treatment.

2.1.7 Experiment 7

The purpose of Experiment 7 was to compare directly the antifungal efficacy of Novexatin® (NP213), NP339 and Terbinafine® (a marketed antifungal agent) in the ex vivo nail system. When Novexatin® (NP213), NP339 and Terbinafine® were applied to the dorsal surface of infected nails, treatment with Novexatin® (NP213) and NP339 for either 14 d or 28 d resulted in no recoverable fungal growth at both time points (FIG. 9). However, treatment with Terbinafine® was unsuccessful after both 14 d and 28 d of treatment.

2.1.8 Experiment 8

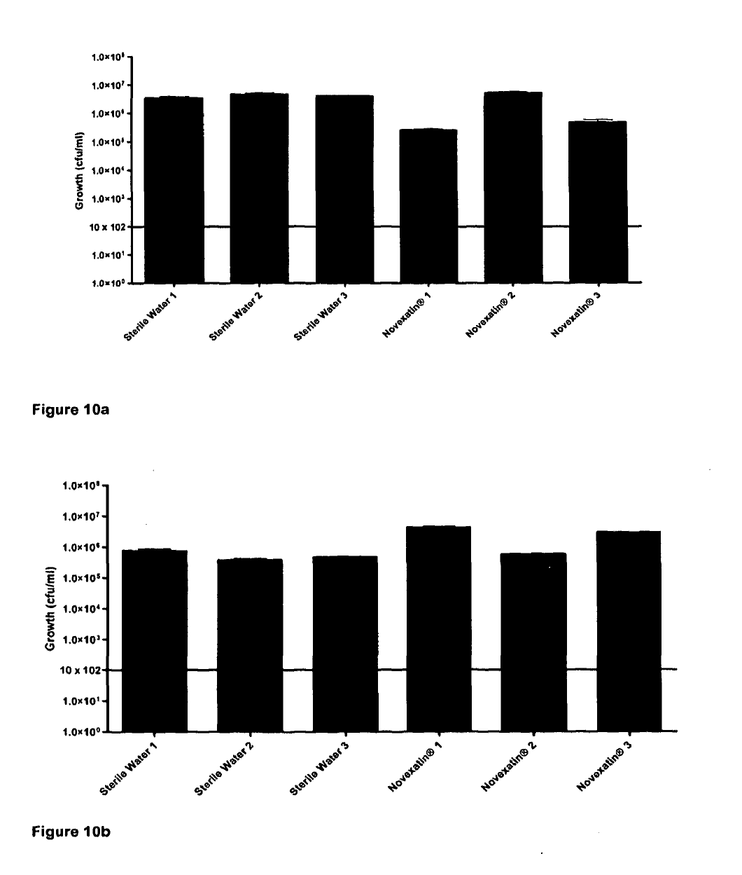

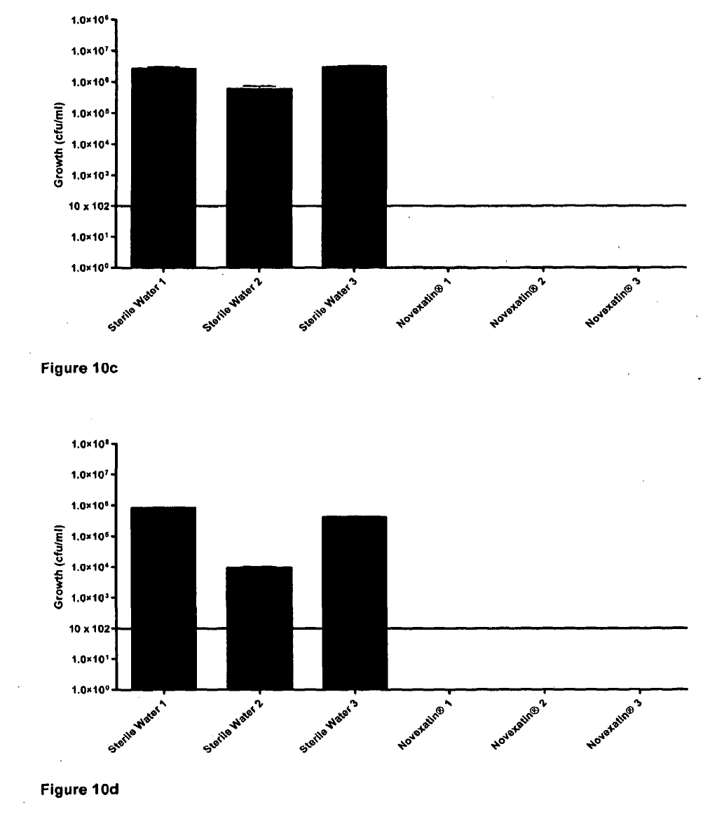

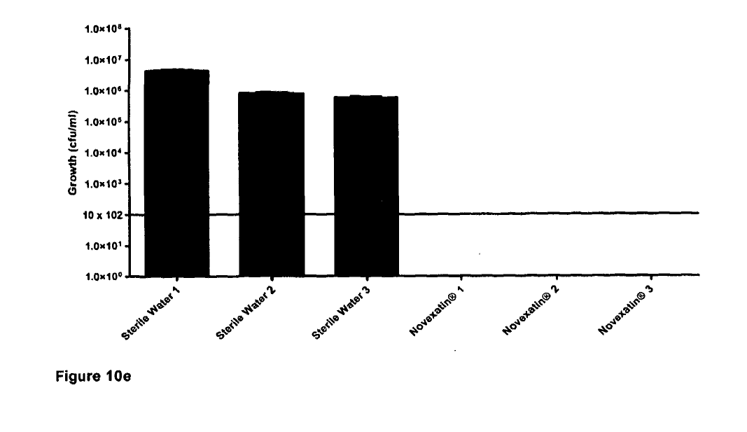

The purpose of Experiment 8 was to compare the preventative efficacy of Novexatin® (NP213) in the ex vivo nail model. After 24 hours, no fungal growth was evidenced for any of the nail fragments which had been pre-treated by Novexatin® (NP213). No fungal growth was observed up to 14 days after contact of the nail fragment with the spore suspension (see FIG. 10).

2.2 Visual Evidence of Fungal Clearance from Nails Treated with Novexatin® (NP213)

Prior to processing nail fragments after 28 d treatment for the determination of fungal survival in Experiment 2, photographs were taken of nails treated with Novexatin® (NP213) and nails treated with vehicle alone (50% (w/v) PEG 8000+20% (w/v) Urea) (FIG. 7). From the nail fragments treated with vehicle alone, it is clear that the nails are infected with T. rubrum as indicated by the clear white “fluffy” fungal biomass. Nails treated with Novexatin® (NP213) show no visual evidence of fungal infection indicating clearance of the surface infection. This result is backed by data presented in FIG. 2 on isolation of fungal colonies.

### 3. Discussion

The effects of the Novexatin® (NP213) formulation on the survival of fungi on nails are presented here, primarily as the numbers of cfu recovered from the nails after treatment (FIGS. 1-6). The effects of the treatments were also clear when the nails were examined visually, as shown in FIG. 7. In addition, the preventative effect of Novexatin® (NP213) was tested.

It was clear (FIG. 1) that treatment of infected nail fragments with 10% (w/v) Novexatin® (NP213) in a 50% (w/v) PEG 8000 vehicle resulted in the isolation of no live fungal colonies after 28 d of treatment, indicating successful killing of T. rubrum NCPF0118 (>99.9%) in this ex vivo nail model. The addition of 20% (w/v) urea to the vehicle containing 5% (w/v) Novexatin® (NP213) resulted in the isolation no fungi, whereas in the absence of urea a small number of fungi were successfully isolated. The addition of 20% (w/v) urea to the vehicle containing 1% (w/v) Novexatin® (NP213) resulted in the isolation of fewer fungi than in vehicle alone, albeit at levels that were not statistically significant.

The next experiment (FIG. 2) confirmed the antifungal effect of Novexatin® (NP213) in the 50% (w/v) PEG 8000 vehicle containing 20% (w/v) urea. As also demonstrated in FIG. 1, treatment with 10% (w/v) Novexatin® (NP213) reduced the fungal burden of the nails to levels below the limit of detection and significant reductions in fungal survival were observed when treated with 2.5% and 5.0% (w/v) Novexatin® (NP213). Even on nails treated on alternate days with 2.5%, 5.0% and 10.0% (w/v) Novexatin® (NP213), the reduction in fungal survival was significant, albeit more surviving fungi were isolated than on nail fragments treated every day. Thus, reducing the application frequency to every other day had in general, only a slight effect. Overall, it would seem that occasional failure to apply the treatment would be unlikely to influence its effectiveness.

Analysis of the effects of Novexatin® (NP213) on the survival of four clinical isolates of Trichophyton spp. indicated that treatment was as effective as that observed when T. rubrum NCPF0118 was used. In vitro MIC testing demonstrated that these strains were less susceptible to Novexatin® (NP213)¹, when compared to T. rubrum NCPF0118. However, this difference in activity is not reflected in human nail tissue, which should be considered a more appropriate model for activity testing for antimicrobials of this type.

It was also demonstrated (FIG. 4) that simulated washing of the nails prior to treatment with Novexatin® (NP213) did not reduce the antifungal efficacy. However, in order to prevent contamination of the test system, the washing process used in this experiment was limited. Full evaluation of the impact of washing the nails will only be achieved during clinical trials. Interestingly, the washing process resulted in slightly enhanced recovery of the test strain from controls, not treated with Novexatin® (NP213), suggesting that washing may enhance growth or viability of the fungi by removing inhibitory metabolic products.

The comparison of Novexatin® (NP213) and fluconazole at dose-for dose equivalent concentrations in relation to the MICs of the two compounds (FIGS. 5 and 6), suggests that Novexatin® (NP213) is more effective than fluconazole at reducing the survival of T. rubrum NCPF0118 in this ex vivo nail model.

The comparison of Novexatin® (NP213), NP339, Itraconazole and Terbinafine against Candida suggests that Novexatin® (NP213) and NP339 are more effective than Itraconazole® and Terbinafine® at reducing the survival of Candida in this ex vivo nail model (see FIGS. 8 and 9).

The nail model of the present invention is able to test the efficacy of potentially therapeutic compounds in the prevention of fungal nail infections, as well as the treatment of fungal nail infections (see.

The experimental system has the ability to show differences in effectiveness of different drugs suggesting potential value as a screening method for antifungals for this application.

### 4. Conclusions

The results of the experiments described here demonstrate the ability of the test compounds, Novexatin® (NP213) and NP339 to dramatically reduce, to a level below that quantifiable using standard microbiological culture methods, the survival of strains of Trichophyton spp. associated with tinea infections, including tinea unguium and strains of Candida. Antifungal activity was maintained in the presence of other potential components of the proposed formulation for topical application during clinical trials. Simulated daily “washing” of the nails did not adversely affect treatment effectiveness. When both Novexatin® (NP213) and fluconazole were tested using the nail model, Novexatin® (NP213) was found to be more effective than fluconazole in eliminating T. rubrum NCPF0118 from nails. The comparison of Novexatin® (NP213), NP339, Itraconazole and Terbinafine against Candida suggests that Novexatin® (NP213) and NP339 are more effective than Itraconazole® and Terbinafine® at reducing the survival of Candida in this ex vivo nail model (see FIGS. 8 and 9). Finally, the ex vivo nail model provides an accurate indication of whether a composition will be effective in the prevention of a fungal nail infection as well as the treatment of such (see FIG. 10).

The ex vivo model of the present invention provides a far more accurate indication of whether a composition is likely to be effective in the treatment or prevention of a fungal nail infection as it replicates the conditions under which the composition will be used. Known methods of testing the efficacy of potentially therapeutic compositions tend to underestimate the in vivo efficacy of compositions. In contrast, the ex vivo model of the present invention accurately predicts the in vivo behaviour of potentially therapeutic compositions.

Improvements and modifications may be incorporated herein without deviating from the scope of the invention.

1. A method of infecting a nail fragment with a fungus comprising the steps of:
obtaining a fungal sample comprising at least 1×10² fungal colony forming units (cfu) per ml;
infecting a nail fragment with the fungal sample by applying the fungal sample to at least a surface of the nail fragment and incubating the nail fragment for an incubation period,
wherein the infected nail comprises at least 1×10² fungal colony forming units (cfu) per cm² of the infected surface(s).

2. The method as claimed in claim 1, wherein the fungus is a dermatophyte, a non-dermatophyte mould or a pathogen.

3. The method as claimed in claim 2, wherein the fungus is a dermatophyte selected from the group consisting of tinea unguium, tinea corporis, tinea capitis, tinea cruris, tinea faciae, tinea pedis, Trichophyton rubrum, Trichophyton mentagrophytes, Trichophyton violaceum, Trichophyton interdigitale, Trichophyton tonsurans, Trichophyton soudanense or Trichophyton verrucosum, Trichophyton schoenleinii, Epidermophyton floccosum, Microsporum gypseum, Microsporum audouinii and Microsporum canis.

4. The method as claimed in claim 2, wherein the fungus is a yeast selected from the group consisting of Candida albicans, Candida krusei, C. Glabrata, C. Famata, C. Parapsilosis, C. Tropicalis, C. Sake, Malassezia furfur and Trichosporon spp.

5. The method as claimed in claim 2, wherein the fungus is a non-dermatophyte mould selected from the group consisting of Acremonium spp, Alternaria spp., Arthrographis kalrae, Aspergillus spp., Arthroderma tuberculatum, Bipolaris spp., Botryodiplodia theobromae, Chrysosporium (Geomyces) pannorum, Cladosporium spp., Fusarium spp, Geotrichium candidum, Nattrassia spp., Onychocola canadensis, Paecilomyces spp., Penicillium spp., Phyllostricta sydowii, Pyrenochaeta unguis-hominis, Scopulariopsis brevicaulis, Scytalidium spp., Synchephalastrum racemosum, Trichoderma spp. and Ulocladium spp.

6. The method as claimed in claim 1, wherein the fungal sample comprises at least 1×10⁴ fungal colony forming units (cfu) per ml.

7. The method as claimed in claim 1, wherein the fungal sample is applied to the ventral side of the nail only.

8. The method as claimed in claim 7, wherein the infected nail has a ventral side comprising at least 1×10² fungal colony forming units (cfu) per cm², and a dorsal side comprising less than 5 fungal cfu per cm².

9. The method as claimed in claim 1, wherein the nail fragment is incubated at temperatures of 25 to 40 degrees Celsius at a humidity level of 50% for up to 50 days.

10. A method of assessing the efficacy of potential therapeutic compounds in the treatment of a fungal nail infection comprising the step of:
a) identifying at least one potentially therapeutic compound,
b) obtaining a first nail fragment having a first surface, said first surface having a known number of fungal cfu per unit surface area being at least 1×10² per cm²;
c) applying the potentially therapeutic compound to a surface of the nail fragment;
d) assessing the number of fungal cfu per unit surface area of the first surface following application of the potentially therapeutic compound;
e) obtaining a second nail portion, said second nail portion having approximately the same thickness as the first nail portion wherein a first surface of the second nail fragment has approximately the same number of fungal cfu per unit surface area as the first surface of the first nail portion;
f) applying a control composition to a surface of the second nail fragment;
g) assessing the number of fungal cfu per unit surface area of the first surface of the second nail portion following application of the control composition;
h) comparing the number of fungal cfu per unit surface area of step d) and step g) wherein a number of fungal cfu per unit surface area of step d) lower than that of step g) is indicative of a compound effective in the treatment of the fungal nail infection.

11. The method of claim 10, wherein the infected nail fragment is prepared according to the method of any one of claims 1 to 9.

12. The method as claimed in claim 10, wherein steps b) to g) are repeated at least ten times before step h) is performed.

13. The method as claimed in claim 10, wherein a cfu per unit surface area of step d) at least 10% less than that of step g) is indicative of a compound effective in the treatment of the fungal nail infection.

14. A method of assessing the efficacy of potentially therapeutic compounds in the prevention of a fungal nail infection comprising the step of:
a) identifying at least one potentially therapeutic compound,
b) obtaining a first nail fragment having a first surface, said first nail fragment being uninfected with a fungal nail infection;
c) applying the potentially therapeutic compound to the first surface of the first nail fragment;
d) applying a fungal sample to a surface of the first nail fragment, said fungal sample comprising at least 1×10² fungal colony forming units per ml;
e) determining the number of fungal cfu per unit surface area of the first surface of the first nail fragment;
f) obtaining a second nail fragment, said second nail fragment having approximately the same thickness as the first nail fragment, wherein the second nail fragment is uninfected with a fungal nail infection;
g) applying a control composition to the first surface of the second nail fragment;
h) applying the fungal sample to a surface of the second nail fragment, said fungal sample comprising at least 1×10² fungal colony forming units per ml;
i) determining the number of fungal cfu per unit surface area of the first surface of the second nail fragment;
j) comparing the number of fungal cfu per unit surface area of step e) and step g) wherein a number of fungal cfu per unit surface area of step e) lower than that of step g) is indicative of a compound effective in the prevention of the fungal nail infection.

15. The method as claimed in claim 14, wherein the potentially therapeutic compound is applied to the ventral side of the first nail fragment and the control composition is applied to the ventral side of the second nail fragment.

16. The method as claimed in claim 15, wherein the thickness of the first and second nail portions are the same to within 1%.

17. The method as claimed in claim 16, wherein the potentially therapeutic compound is applied to the nail fragment in the form of a potentially therapeutic composition comprising one or more pharmaceutical excipients.

18. The method as claimed in claim 17, wherein the control composition consists of same components as the potentially therapeutic composition, absent the potentially therapeutic compound.

19. The method as claimed in claim 18, wherein the reduction of the number of fungal cfu after application of the control composition is less than 0.5%.

20. The method as claimed in claim 10, wherein the fungal nail infection is distal subungual onychomycosis, white superficial onychomycosis, proximal subungual onychomycosis or candidal onychomycosis.

21. The method as claimed in claim 14, wherein step c) and step g) are repeated once a day for 1 to 7 days.

22. The method as claimed in claim 14, wherein a cfu per unit surface area of step e) at least 75% less than that of step g) is indicative of a compound effective in the prevention of the fungal nail infection.


SEQUENCE LISTING (3 sequences)

SEQ ID NO: 1 - protein, 7 aa, Artificial Sequence
  synthetic peptide
RRRRRRR

SEQ ID NO: 2 - protein, 13 aa, Artificial Sequence
  synthetic peptide
RRRRRRRRRRRRR

SEQ ID NO: 3 - protein, 200 aa, Artificial Sequence
  synthetic peptide
RRRRRRRXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXX